(19) United States
(12) Patent Application Publication
(10) Pub. No.: US 2025/0285713 A1
(43) Pub. Date: Sep. 11, 2025

(54) Catalyst Inverse Optimization and Transferable Descriptor Identification Strategy

(71) Applicant: GreenCat LLC, Lewes, DE (US)

(72) Inventor: Shambhawi Shambhawi, London (GB)

(73) Assignee: GreenCat LLC, Lewes, DE (US)

(21) Appl. No.: 19/068,035

(22) Filed: Mar. 3, 2025

Related U.S. Application Data
(60) Provisional application No. 63/562,737, filed on Mar. 8, 2024.

(51) Int. Cl. G16C 20/10; G16C 20/20; G16C 20/70

(52) U.S. Cl. CPC G16C 20/10; G16C 20/20; G16C 20/70

(57) ABSTRACT

The present invention is a method and device for simplification of interrelated complexities in a reaction relates to inverse optimization which relates to an algorithm which can potentially determine generalized descriptor values for various reaction systems, spanning pharmaceutical, polymeric, and other industries such as automobiles, specialty chemicals, and cosmetics. The invention lies in the hypothetical catalyst optimization step, and the core of the invention involves descriptor identification through hypothetical optimization in intricate systems like reaction networks. The aim of the optimization is to identify transferable descriptor and their value ranges to high performing catalyst solutions (i.e., activity, selectivity and stability). These value ranges will later be used for screening real materials with required catalytic performance.

The present application claims priority under 35 USC 119(e) to U.S. Provisional Application No. 63/562,737 filed Mar. 8, 2024, the entire contents of which are incorporated by reference in its entirety.

### FIELD OF THE INVENTION

The present invention relates to the field of catalyst design and optimization using and/or based on descriptor identification and inverse design strategy. Its applicability spans diverse industries including the pharmaceutical, petrochemical, ammonia production reactions, catalytic biofuel production, automobiles and cosmetics industries.

### BACKGROUND OF THE INVENTION

Catalyst design approaches have always been direct, i.e. catalytic performance of a material is enhanced through changes in catalyst's composition and structure, with evidence of improved performance collected via experiment or prediction models. Forward exploration of potential catalysts is a very inefficient way of developing new materials: the limitations of synthesis, experimental and quantum chemical calculation techniques allow one to explore only a small fraction of the potentially available ‘chemical space’ including possible materials, the combinations of atoms, and variations in structures, etc.

The literature on catalysis reports suggests that there are many instances where enhanced catalytic performance can be reproduced by a different material with a similar reaction energy profile, for example, there are reports that NiSn[8] and NiB[2] systems for CH₄ dry reforming reaction give similar results. Both catalytic systems have a lower binding affinity towards C* relative to Ni, thus demonstrating stability against carbon deactivation. Additionally, the activation barriers for CHx oxidation are higher, which in turn limits their activity. Similarly, there can be multiple solutions to a catalyst design problem if the focus is on achieving an acceptable performance that is significantly better than the existing industrial catalyst. This can be achieved by identifying relevant surface energies and locating the optimum value for maximum performance, i.e. via a descriptor-based screening methodology.

Descriptor-based screening is a quick approach to screen materials that likely delivers better catalytic performance based on a volcano plot for a simple reaction system. However, in a study reported in Mohan et al., it is suggested that the identified descriptors, i.e. relevant reaction energies, might not be transferable to different catalyst surfaces in the case of a complex reaction system like CO₂ methanation. This suggests that creating a generalizable descriptor searching process may be the most important step to develop a screening tool.

In Shambhawi et al., they describe and introduce a workflow based on network science that can create a transferable partial reaction network (PRN). The PRN is uses a set of relevant reaction steps and intermediates to evaluate and compare the minimum reactant conversions over different catalyst surfaces. However, the methodology that employs evaluating a PRN is still prohibitively expensive if one's aim is to perform a quick preliminary screening.

Previously, Rangarajan et al. had introduced an inverse optimization technique for hypothetical catalysts that can be used to circumvent the computational bottleneck that come from more complex screening. However, such strategies are extremely difficult to implement for at least the following two reasons:
(1) there are thermodynamic and equilibrium constraints in a reaction that cannot be violated,
(2) the number of optimization variables are significant and finding a real catalyst with exact same energies is unlikely if not impossible.

It would be advantageous if one could address the above two concerns by employing one or more of the following:
(1) Incorporating equilibrium constraints and reaction thermodynamics into an optimization methodology,
(2) Adding both thermodynamic and scaling relation constraints between intermediates and transition state species of the reaction into the optimization methodologies.
(3) Identifying the relevant reaction descriptor(s) and its/their corresponding values from sensitivity and reaction pathway analysis of the optimized reaction energies of the hypothetical catalyst solutions, and/or
(4) Using the descriptor value ranges observed in the list of optimized catalyst solutions as a tolerance for a quick preliminary screening of new catalyst materials.

It is with these limitations and desired actions that the present action was developed.

### BRIEF SUMMARY OF THE INVENTION

The present invention relates to a method and device for simplification of interrelated complexities in a reaction system. In an embodiment, the present invention utilizes inverse optimization and an algorithm that can potentially determine generalized descriptor values for various reaction systems, and its use should lead to uses spanning the pharmaceutical, polymeric, and other industries, including but not limited to industries such as the automobile, specialty chemicals, and cosmetics industries. In an embodiment, the present invention also relates to the use of a hypothetical catalyst optimization step, and in a variation of the invention, it involves descriptor identification through hypothetical optimization in intricate systems like reaction networks.

The present invention is able to solve some of the issues of the prior art by:
(1) Incorporating equilibrium constraints and reaction thermodynamics into an optimization methodology,
(2) Adding both thermodynamic and scaling relation constraints between intermediates and transition state species of the reaction into the optimization methodologies.
(3) Identifying the relevant reaction descriptor(s) and its/their corresponding values from sensitivity and reaction pathway analysis of the optimized reaction energies of the hypothetical catalyst solutions, and/or
(4) Using the descriptor value ranges observed in the list of optimized catalyst solutions as a tolerance for a quick preliminary screening of new catalyst materials.

### BRIEF DESCRIPTION OF THE SEVERAL VIEWS OF THE DRAWING

FIG. 1 shows descriptor value distribution observed over the screened optimized hypothetical catalyst solutions.

FIG. 2 shows scaling relations (a) C*—CH* and (b) C*—CHO* developed over Ni-based catalysts for relevant intermediate binding energies.

### DETAILED DESCRIPTION OF THE INVENTION

The present invention relates to a method and device for simplification of interrelated complexities in a reaction and it relates to inverse optimization which uses an algorithm that can potentially determine generalized descriptor values for various reaction systems. The present inventions uses should be able to span industries such as the pharmaceutical, polymeric, and other industries, including but not limited to industries such as the automobile, specialty chemical, and cosmetics' industries. In an embodiment, the invention uses a hypothetical catalyst optimization step, and alternatively and/or addition, the invention involves descriptor identification through hypothetical optimization in intricate systems like reaction networks.

In an embodiment, the invention comprises an intelligent strategy of optimizing dynamic mass flow through reaction networks in hypothetical catalysts followed by descriptor identification from a list of optimized solutions and inverse screening of real catalysts based on descriptor value ranges. In one aspect, the invention comprises one or more of the following steps:
a) Evaluating catalyst performance by constructing a micro-kinetic model (MKM) based on a comprehensive reaction network using reaction energetics obtained over an initial catalyst surface. The initial catalyst surface can be a known active metal system whose catalytic performance needs to be enhanced. The details on developing a comprehensive micro-kinetic model based on energetics obtained from quantum chemistry computation can be found using methodologies as proposed by Mohan et al. (see references 1 and 2 below). Micro-kinetic models are used in catalyst investigation studies to obtain relative comparisons of catalyst performance.
b) Performing a reaction energy optimization using hypothetical catalysts that are bound by thermodynamic and scaling relation constraints. This could either mean maximizing reaction conversion x_{R}_{ᵢ}, product yield y_{P}_{ᵢ}, selectivity χ_{P}_{ᵢ}, coverages of deactivating intermediates θᵢ or any combination or all of these factors.

$${Z}_{cat} = max ( {x}_{{R}_{i}} , {y}_{{P}_{i}} , {χ}_{{P}_{i}} , 1 - {θ}_{i} )$$

Herein, the decision variables are energies corresponding to reaction species, i.e. intermediates and transition states. These energies can be obtained from Eᵢ^{QC} that corresponds to the total energies (obtained via any quantum chemistry computation technique) of individual intermediate and transition state species observed over the initial catalyst. Any changes in these individual energies are defined as the decision variable(s) (πᵢ).

$${E}_{i} = {E}_{i}^{QC} + {π}_{i}$$

wherein i corresponds to the intermediate or transition state species.

Unlike previous optimization approaches (for example, as described by Rangarajan et al. (see reference 3), scaling relation constraints were also included in the present optimization. Based on these relations, the individual energy changes were written in terms of change in various species' total energy of adsorbed atomic species, π_{Z}, and on the slopes of the linear relations for both intermediate and transition states, γ_{z}(x),αⱼ), see Equations 3-5 below.

$${E}_{z} = {E}_{Z}^{QC} + {π}_{Z} \qquad ( 3 )$$

$${π}_{i} = {\sum }_{Z} {γ}_{Z} ( x ) {π}_{Z} \qquad ( 4 )$$

$${π}_{TS , j} = {{α}_{j} ( {\sum }_{Z} {γ}_{Z} ( x ) {π}_{Z} )}_{j} \qquad ( 5 )$$

where j corresponds to the elementary reaction number. The corresponding atomic species Z of an intermediate/transition states species are the atoms of the species through which they bind to the metal surface.

### Demonstration of the Optimization Technique:

The hypothetical catalyst optimization is demonstrated for the CH₄ dry reforming reaction system using Ni (111) as the catalyst in an initial guess. For this example system, there are a total of 55 (20 intermediates, 35 transition states) decision variables. These decision variables are continuous within bounds and are subject to or under the following constraints as shown in 6-10:

$${E}_{TS , j} \geq {E}_{IS , j} \qquad ( 6 )$$

$${E}_{TS , j} \geq {E}_{FS , j} \qquad ( 7 )$$

$${γ}_{Z} ( {x}_{max} ) < {γ}_{Z} ( {x}_{i} ) < {γ}_{Z} ( {x}_{0} ) \qquad ( 8 )$$

$$- 50 \leq {π}_{Z} \leq 50 \qquad ( 9 )$$

$$- 2 \leq {γ}_{z} ( x ) , {α}_{j} \leq 2 \qquad ( 10 )$$

The constraints in Equations 6 and 7 are nonlinear as the changes in transition state energies, π_{TS,j}, are given by Equation 5 wherein the slope αⱼ, corresponding to a Bronsted-Evans-Polanyi (BEP) relation, and γ_{z}, thermodynamic scaling relations, which are multiplied to get the change in transition state energy. The bounds in Equation 8 are based on values reported in the literature. (see Montemore et al.)

The reaction conditions, inlet conditions and zero-point energies of species on the initial catalyst surface were kept constant during optimization. The optimization was performed for the CH₄ dry reforming reaction in a plug flow reactor with a reaction temperature of 973 K, 10 bar pressure, space time 0.01 g·h·mol-1 and inlet feed ratio of 1:1 for CH₄:CO₂. MATLAB's pattern search optimization toolbox was used to minimize the objective functions i.e., min [symbol] θ_{C}*) for initial guess Ni (111). Here, x(CH₄) is the fractional conversion of CH₄ and θ_{C}* is the surface carbon coverage obtained from the microkinetic model.

The optimization resulted in a total of 1095 optimized hypothetical instances of catalysts that satisfied the tolerance for minimum carbon optimization.

This allowed the identification of problem-specific reaction descriptors and their required value ranges via sensitivity analysis and reaction pathway analysis on the initial catalyst guess and the optimized hypothetical catalysts respectively.

### Demonstration of the Descriptor Identification Technique:

All of the 1095 hypothetical catalyst solutions that were previously noted in the optimization step, had the same dominant pathway, i.e. CH₄(g)-->CH₃*-->CH₂*-->CH*-->CHO*-->CO*-->CO(g). The Rate Determining Step (RDS) being CH* oxidation to HCO*. Upon sensitivity analysis performed over Ni (111) surface, it was found that C* surface coverage was most sensitive to carbon binding energy with a normalized sensitivity coefficient of 1. This was followed by the CH* oxidation barrier to HCO* whose sensitivity coefficient is 0.34. A positive sensitivity coefficient suggests that the coverage decreases with a decrease in the descriptor value, whereas a negative value suggests a decrease with an increase in the descriptor value.

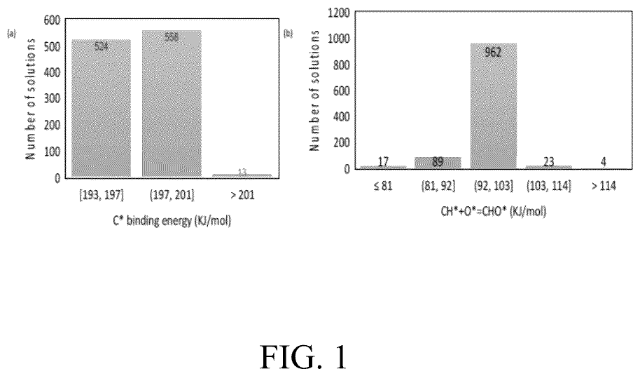

Previously reported studies similarly suggested that increasing the binding energy of carbon can help with catalyst stability, although this also leads to a lower activity. For example, this is the case with NiB[2] and NiSn.[8]. Chen et. al. compared the reactants' activation barriers against C binding energy and affirmed the above inverse relationship between activity and stability based on C binding energy for a range of Ni based catalysts.[see reference 8] Therefore, for this invention, the C binding energy was maintained at ±10 kJ/mol and the next most sensitive reaction energy was used as the descriptor, i.e., the CH* oxidation barrier to HCO*. In an embodiment, the barriers corresponding to the Rate Determining Step (RDS) observed over the hypothetical can also be used as a descriptor. In this case, however, it is same as the sensitive reaction energy observed over the initial catalyst surface (after C binding energy). FIG. 1 shows the range of values observed for this calculation in the selected solutions.

FIG. 1 shows the descriptor value distribution observed over the screened optimized hypothetical catalyst solutions. For comparison purposes, the initial C* binding energy and CH* oxidation barrier on the initial guess Ni (111) were 192 kJ/mol and 146 kJ/mol, respectively.

In an embodiment, through hypothetical catalyst optimization, real catalysts can be screened using the identified descriptor and its value range. This can be done by developing linear prediction models, such as scaling relations, or non-linear prediction models using machine learning. The nature of the screening model depends on the search space that the user will/can define. The approach of screening real catalysts, once the descriptor and value range are identified, is called inverse catalyst design, which is one embodiment of the present invention.

### Demonstration of Catalyst Screening:

One or more catalysts for the above-mentioned example system can be screened based on the identified descriptor and value ranges, i.e., a CH* oxidation barrier can be set to <116 KJ/mol. This can be done by developing linear prediction models, such as scaling relations, or by non-linear prediction models using machine learning. In one embodiment, the invention is focused on metallic systems (mostly those metals that come from the d-block), wherein scaling relations are found to be simpler and more efficient. Accordingly, in this aspect of the invention, scaling relations are reported for bulk systems.

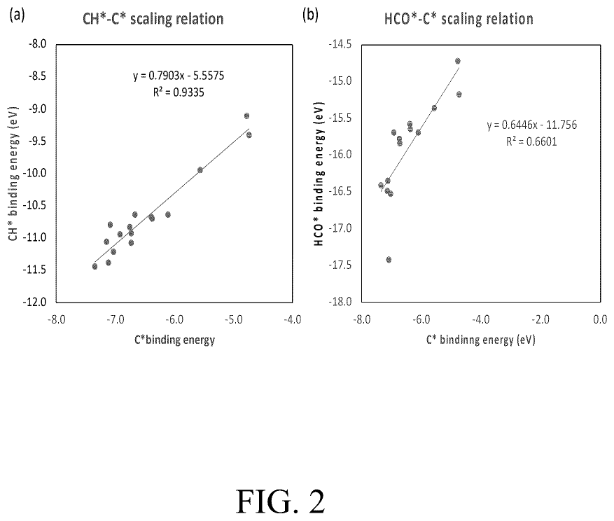

FIG. 2 shows the scaling relation developed using the Ni-based systems. These are the thermodynamic scaling relations between C*—CH* binding energies (MAE of 0.14 eV) and C*—HCO* binding energies (MAE of 0.26 eV). A linear scaling relation is also found to exist between the (C*,O*)—CH* binding energies with an MAE of 0.23 eV, see Equation (33) below. It performs slightly better than the C*—HCO* relation.

FIG. 2 shows a scaling relations (a) C*—CH* and (b) C*—CHO* developed over Ni-based catalysts for relevant intermediate binding energies using the below formula.

$${E}_{{HCO}^{*}} = 0.2669 {E}_{{C}^{*}} + 0.4285 {E}_{{O}^{*}} \qquad ( 33 )$$

Based on the scaling relations and their C and O binding energies, several catalysts were selected for evaluating transition state barriers. From the optimized hypothetical catalysts for minimum carbon coverage, it was observed that catalysts with similar C binding energy such as Ni(111) (□10 kJ/mol) and CH oxidation barrier lower than 116 kJ/mol (at least 30 kJ/mol lower from that observed over Ni(111)) demonstrated greater activity and stability. As to the BEP relationships, without being bound by theory it is postulated that lower activation barriers can be a consequence of lower O binding energy since the transition state is more stabilized. Therefore, bulk materials with C binding energies similar to Ni(111) and O binding energy slightly lower than Ni(111) were selected, for example, the putative catalysts. Ni₃Fe and Ni₃Si.

After a transition state investigation over the selected bulk materials, an activation barrier of less than 116 kJ/mol was observed for Ni₃Si. Similarly, a 50 kJ/mol barrier was found to be reported for Ni₃Fe in the literature. [See reference 12] The computational study based on density functional theory (DFT—a quantum chemistry tool for computational investigation) and experimental study over Ni₃Fe further confirmed the higher activity and stability of the catalyst. The following results calls for further investigation into similar catalysts like Ni₃Si.

In one embodiment of the invention, after the descriptor based primary screening, a secondary screening of catalyst materials can be performed. This will be done by screening materials using scaling relations or a machine learned prediction model and then further comparing catalyst performances of screened materials using transferable partial reaction networks constructed using guidelines provided as described by Shambhawi et al.

In an embodiment, the approach on inverse catalyst design and optimization can be performed by identifying a descriptor that is an identifier of a reaction, e.g., reactants, products and reaction conditions. In a variation, the descriptor is not an identifier of the catalyst and will not change if the catalyst surface changes. For a given set of reaction conditions, initial catalyst guesses and a range of reactor space-time, the descriptor is identified in the following stepwise manner:
a) Evaluating catalyst performance by constructing a micro-kinetic model (MKM) based on a comprehensive reaction network using reaction energetics obtained over an initial catalyst surface. The initial catalyst surface can be a known active metal system whose catalytic performance needs to be enhanced. Details on developing a comprehensive micro-kinetic model based on energetics obtained from quantum chemistry computation can be found in references 1 and 2, listed below. MKM models are used in catalyst investigation studies for relative comparison of catalyst performances.
b) Performing a reaction energy optimization using hypothetical catalysts that are bound by thermodynamic and scaling relation constraints. This could either mean maximizing reaction conversion x(Rᵢ), product yield y(Pᵢ), selectivity χ(Pᵢ), coverages of deactivating intermediates θᵢ or all of them.

$${Z}_{cat} = max ( {x}_{{R}_{i}} , {y}_{{P}_{i}} , {χ}_{{P}_{i}} , 1 - {θ}_{i} ) \qquad ( 1 )$$

Herein, the decision variables are energies corresponding to reaction species, i.e. intermediates and transition states. These energies can be obtained using Equation 2 below, where Eᵢ^{QC} corresponds to the total energies (obtained via any quantum chemistry computation technique) of individual intermediate and transition state species observed over the initial catalyst. Any changes in these individual energies are defined as the decision variables (7i).

$${E}_{i} = {E}_{i}^{QC} + {π}_{i} \qquad ( 2 )$$

where, i corresponds to the intermediate or transition state species.

Unlike the previous optimization approach as described in reference 3 below, scaling relation constraints are included in the present optimization. Based on these relations, the individual energy changes were written in terms of change in species total energies of adsorbed atomic species, π_{Z}, and slopes of the linear relations for both intermediate and transition states, (γ_{z}(x),αⱼ), see Equations 3-5.

$${E}_{z} = {E}_{Z}^{QC} + {π}_{Z} \qquad ( 3 )$$

$${π}_{i} = {\sum }_{Z} {γ}_{Z} ( x ) {π}_{Z} \qquad ( 4 )$$

$${π}_{TS , j} = {{α}_{j} ( {\sum }_{Z} {γ}_{Z} ( x ) {π}_{Z} )}_{j} \qquad ( 5 )$$

where j corresponds to the elementary reaction number. The corresponding atomic species Z of an intermediate/transition states species are the atoms of the species through which it is binding to the metal surface.

A sample implementation of this optimization approach is presented supra.

The optimization outputs several solutions with required catalyst performances. The most sensitive variables, e.g., energies, observed on the initial catalyst guess and the optimized hypothetical catalysts respectively are the identified descriptors. The value range observed for these descriptors in the list of optimized solutions is also noted and a descriptor value range is defined.

Through hypothetical catalyst optimization, real catalysts can be screened using the identified descriptor and its value range. This can be done by developing linear prediction models, such as scaling relations, or non-linear prediction models using machine learning. The nature of the screening model depends on the search space that the user will define. This approach of screening real catalysts, once the descriptor and value range are identified, is called inverse catalyst design.

After the descriptor based primary screening, a secondary screening of catalyst materials can be performed. This can be done by comparing catalyst performances of screened materials using transferable partial reaction networks constructed using guidelines provided in reference 4 listed below.

In an embodiment, the present invention relates to a method of designing and/or optimizing a catalyst design in a reaction, said method comprising:
a) Identifying a descriptor that is an identifier of the reaction in the absence of the catalyst wherein the identifier is selected from the group consisting of a reactant, a product, and one or more conditions in said reaction;
b) Selecting a plurality of example catalysts;
c) Evaluating the plurality of example catalysts using a micro-kinetic model, which uses reaction energetics obtained over a surface of the plurality of example catalysts;
d) Performing a reaction energy optimization using quantum chemistry computations using thermodynamic and/or scaling relation constraints;
e) Identifying the catalyst the performs best in the reaction energy optimization.

In a variation, the method is performed using a computer. In a variation, the method uses artificial intelligence. In a variation, the one or more conditions is one or more members selected from the group consisting of solvent choice, pH, temperature, pressure, time, light exposure, stirring rate, surface area of reactants, and presence or absence of inhibitors.

In a variation, at least two or more conditions are used. In a variation, at least three or more conditions are used.

In a variation, the method further comprises ascertaining a value range for a descriptor and using the value range to perform the reaction energy optimization.

In a variation, the reaction energy optimization comprises one or more of maximizing reaction conversion, product yield, selectivity, or coverages of deactivating intermediates.

In a variation, the method uses all of maximizing reaction conversion, product yield, selectivity, and coverages of deactivating intermediates. In a variation, scaling relation constraints are used.

In a variation, calculating the reaction energy optimization comprises one or more of calculating a change in species total energies of adsorbed atomic species, and/or calculating slopes of linear relations for both intermediate and transition states.

In a variation, the method uses both calculating the energy optimization comprises one or more of calculating a change in species total energies of adsorbed atomic species, and calculating slopes of linear relations for both intermediate and transition states.

In a variation, the calculating the change in species total energies of adsorbed atomic species, and/or calculating slopes of linear relations for both intermediate and transition states comprises using one or more of the following formulas:

$${E}_{z} = {E}_{Z}^{QC} + {π}_{Z}$$

$${π}_{i} = {\sum }_{Z} {γ}_{Z} ( x ) {π}_{Z}$$

$${π}_{TS , j} = {{α}_{j} ( {\sum }_{Z} {γ}_{Z} ( x ) {π}_{Z} )}_{j} .$$

In a variation, the method further comprises a secondary screening of catalyst materials performed by comparing catalyst performances of screened materials using transferable partial reaction networks. In a variation, the method is performed on a computer.

In a variation, the method uses artificial intelligence.

The following references are incorporated by reference in their entireties:
1. Mohan, O., et al., Investigating CO₂ Methanation on Ni and Ru: DFT Assisted Microkinetic Analysis. ChemCatChem, 2021. 13(10): p. 2420-2433.
2. Mohan, O., et al., Investigating methane dry reforming on Ni and B promoted Ni surfaces: DFT assisted microkinetic analysis and addressing the coking problem. Catalysis Science & Technology, 2020. 10(19): p. 6628-6643.
3. Rangarajan, S., C. T. Maravelias, and M. Mavrikakis, Sequential-Optimization-Based Framework for Robust Modeling and Design of Heterogeneous Catalytic Systems. The Journal of Physical Chemistry C, 2017. 121(46): p. 25847-25863.
4. Shambhawi, J. M. Weber, and A. A. Lapkin, Micro-kinetics analysis based on partial reaction networks to compare catalysts performances for methane dry reforming reaction. Chemical Engineering Journal, 2023. 466: p. 143212.
5. Wang, Z. and P. Hu, Towards rational catalyst design: a general optimization framework. Phil. Trans. R. Soc., 2016. 374(2061): p. 20150078.
6. Baumes, L., et al., Using Artificial Neural Networks to Boost High-throughput Discovery in Heterogeneous Catalysis. 2004. 23(9): p. 767-778.
7. Corma, A., et al., Application of Artificial Neural Networks to Combinatorial Catalysis: Modeling and Predicting ODHE Catalysts. Chemphyschem: a European journal of chemical physics and physical chemistry, 2002. 3(11): p. 939-945.
8. Chen, S., J. Zaffran, and B. Yang, Descriptor Design in the Computational Screening of Ni-Based Catalysts with Balanced Activity and Stability for Dry Reforming of Methane Reaction. ACS Catalysis, 2020. 10(5): p. 3074-3083.
9. Wang, R., et al., Nanocarbon-Based Electrocatalysts for Rechargeable Aqueous Li/Zn-Air Batteries. 2018. 5(14): p. 1745-1763.
10. Greeley, J., Theoretical Heterogeneous Catalysis: Scaling Relationships and Computational Catalyst Design. 2016. 7(1): p. 605-635.
11. Montemore, M. M. and J. W. Medlin, Scaling relations between adsorption energies for computational screening and design of catalysts. Catalysis Science & Technology, 2014. 4(11): p. 3748-3761.
12. Wang, X., et al., Density-functional theory investigation into the role of Fe doping for improving the carbon resistance over Ni3Fe(111) surface in methane reforming with CO2. Applied Surface Science, 2022. 574: p. 151661.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. While the invention has been described in connection with example system thereof, it will be understood by those skilled in the art that various changes in form and details may be made therein without departing from the spirit and scope of the invention. In any event, the invention is defined by the below claims.

We claim:

1. A method of designing and/or optimizing a catalyst design in a reaction, said method comprising:
a) Identifying a descriptor that is an identifier of the reaction in the absence of the catalyst wherein the identifier is selected from the group consisting of a reactant, a product, and one or more conditions in said reaction;
b) Selecting a plurality of example catalysts;
c) Evaluating the plurality of example catalysts using a micro-kinetic model, which uses reaction energetics obtained over a surface of the plurality of example catalysts;
d) Performing a reaction energy optimization using quantum chemistry computations using thermodynamic and/or scaling relation constraints;
e) Identifying the catalyst the performs best in the reaction energy optimization.

2. The method of claim 1, wherein the method is performed using a computer.

3. The method of claim 2, wherein the method uses artificial intelligence.

4. The method of claim 1, wherein the one or more conditions is one or more members selected from the group consisting of solvent choice, pH, temperature, pressure, time, light exposure, stirring rate, surface area of reactants, and presence or absence of inhibitors.

5. The method of claim 4, wherein at least two or more conditions are used.

6. The method of claim 4, wherein at least three or more conditions are used.

7. The method of claim 1, wherein the method further comprises ascertaining a value range for a descriptor and using the value range to perform the reaction energy optimization.

8. The method of claim 1, wherein the reaction energy optimization comprises one or more of maximizing reaction conversion, product yield, selectivity, or coverages of deactivating intermediates.

9. The method of claim 8, wherein the method uses all of maximizing reaction conversion, product yield, selectivity, and coverages of deactivating intermediates.

10. The method of claim 1, wherein scaling relation constraints are used.

11. The method of claim 1, wherein calculating the reaction energy optimization comprises one or more of calculating a change in species total energies of adsorbed atomic species, and/or calculating slopes of linear relations for both intermediate and transition states.

12. The method of claim 11, wherein the method uses both calculating the reaction energy optimization comprises one or more of calculating a change in species total energies of adsorbed atomic species, and calculating slopes of linear relations for both intermediate and transition states.

13. The method of claim 11, wherein the calculating the change in species total energies of adsorbed atomic species, and/or calculating slopes of linear relations for both intermediate and transition states comprises using one or more of the following formulas:
$${E}_{z} = {E}_{Z}^{QC} + {π}_{Z}$$
$${π}_{i} = {\sum }_{Z} {γ}_{Z} ( x ) {π}_{Z}$$
$${π}_{TS , j} = {{α}_{j} ( {\sum }_{Z} {γ}_{Z} ( x ) {π}_{Z} )}_{j} .$$

14. The method of claim 1, wherein the method further comprises a secondary screening of catalyst materials performed by comparing catalyst performances of screened materials using transferable partial reaction networks.

15. The method of claim 13, wherein the method is performed on a computer.

16. The method of claim 15, wherein the method uses artificial intelligence.
